(19) United States
(12) Patent Application Publication
(10) Pub. No.: US 2004/0265266 A1
(43) Pub. Date: Dec. 30, 2004

(54) Use of magnesium hydroxide and calcium compounds with and without a carrier medium to treat animal waste: to reduce air emissions (including ammonia volatilization) from, retain nutrients from, and manage phosphorous solubility of decaying animal litter, manure, and animal excretions and waste in CAFOs and animal enclousures; to reduce farm nutrient runoff; to extract and bind waste nutrients for fertilizer use; and to reduce air emission of waste-based fertilizers and animal bedding

(76) Inventors: Michael Augustus Champ, Falls Church, VA (US); Charles Adams Bakewell, Earlysville, VA (US)

Correspondence Address: Charles A. Bakewell, 1070 Earlysville Forest Drive, Earlysville, VA, 22936, US

(21) Appl. No.: 10/831,410

(22) Filed: Apr. 24, 2004

Related U.S. Application Data
(60) Provisional application No. 60/465,345, filed on Apr. 25, 2003.
(60) Provisional application No. 60/497,839, filed on Aug. 26, 2003.

(51) Int. Cl. C05F 1/00; B01D 53/34; A61L 9/00

(52) U.S. Cl. 424/76.21; 71/15

(57) ABSTRACT

A method for treating CAFO animal litter, manure and waste, reducing volatilization, retaining nitrogen and phosphorous, and reducing soluble phosphorus levels in litter, manure-based compost, and manure-based fertilizers. The method adds magnesium hydroxide (and calcium carbonate or calcium compounds) with or without a carrier medium to animal litter, compost, manure, and manure-based fertilizers as well as pet and zoo animal bedding material to maintain the litter pH at values inhibiting ammonia and hydrogen sulfide volatilization. Calcium compounds added to the litter and waste reduce soluble phosphorus runoff from fields receiving the resulting litter, fertilizer or lagoon water. Application of magnesium hydroxide and calcium compounds in CAFO waste, washwaters and lagoons precipitates a compound recoverable as fertilizer material. The method is effective in poultry, hog, and dairy CAFOs, animal care facilities, pet and zoo enclosures, pastures, agricultural fields, manure storage piles, animal waste treatment, manure-based fertilizer manufacture, and manure-based fertilizer products.

### CROSS-REFERENCE TO RELATED INVENTIONS Provisional Applications for Patent 60/465,345 and 60/497,839

### STATEMENT REGARDING FEDERALLY SPONSORED RESEARCH OR DEVELOPMENT

Not applicable

### REFERENCE TO A SEQUENCE LISTING, A TABLE, OR A COMPUTER PROGRAM LISTING COMPACT DISC APPENDIX

None

### BACKGROUND OF THE INVENTION

Commercial livestock and poultry growing operations keep expanding to meet the demands of domestic and foreign markets for meat and dairy products. The business of animal production is increasingly centered in high volume concentrated animal feeding operations (CAFOs) and employs science-based techniques for optimizing conversion of animal feed into animal protein. Unfortunately, the development and application of innovative methods and technologies for litter and manure management, control of air emissions and farm runoff, and nutrient retention for use in fertilizer by-products have not kept pace.

The majority of producers are still handling manure in much the same way as producers did thousands of years ago when they cleaned their small barns and spread the manure on nearby fields. The volume of manure certainly has increased and the equipment is bigger, but the management process has not been refined very much. Producers and agricultural specialists recognize the nutrient value of manure, both as a fertilizer and a soil conditioner, but the product characteristics have meant that the waste as a CAFO byproduct has little market value. As a result, many producers simply try to dispose of their manure as cheaply and on as small a land area as possible. Some state governments fund transportation programs to help relocate manure from surplus-producing counties to other counties with fields that can absorb the surplus without negative impacts on watersheds. To some extent, animal waste and air emissions are considered a commercial burden and a public nuisance.

Manure management is further complicated by the related emissions generated from the time it is generated and deposited through its collection and storage and its disposition and decomposition. Emissions have become an increasingly urgent problem as economic development and subdivision housing development confront agricultural interests with the priorities and preferences of non-farm residents who do not want to live with the emissions of manure.

Given current economic conditions, least cost disposal may represent a practical approach to poultry manure handling. However, producers must ask whether this approach is sustainable. In this time of growing environmental awareness, producers must balance the effects of economic forces with increasing demands coming from outside the industry. There is strong pressure on all industries to reduce the production and disposal of wastes. Since simple disposal of organic by-products such as manure will become increasingly difficult and expensive, we need to find a better practical approach, perhaps including addition of required minerals to manure to increase its “value” and use as a commercial fertilizer in all forms of agriculture. Another option is the extraction and retention of nutrients from manure in a form suitable for direct, controlled-release or slow-release fertilizer use or augmentation as a designer fertilizer product.

Excess On-Farm Utilization of Manure

There are also growing concerns over the impact that confined animal feeding operations have on water quality and other rural amenities. As a result of domestic and export market forces, technological changes, and industry adaptations, animal production industries have seen substantial changes over the past decade. The number of large confined production units (commonly referred to as Concentrated Animal Feeding Operations (CAFOs) has expanded, and animal production and feed production are increasingly separated geographically. In terms of production, the total number of animal units increased by about 10 percent between 1987 and 1997, while the number of farms decreased by more than half.

Manure is a potential source of water quality degradation from runoff to surface water and leaching to ground water at several stages, including:

Accumulation in open and unpaved feedlots

Storage in holding ponds and lagoons

Uncovered stockpiles

Excess manure and wastewater applied to land

The size and number of animal waste storage lagoons used to store waste before it is applied to fields increases the chance for a leak or a catastrophic break. Over the past several years, major lagoon spills or leaks have been documented in Illinois, North Carolina, Iowa, Kentucky, Minnesota, Missouri, Montana, South Dakota, Utah, Virginia, Washington, and Wisconsin. States such as New York and Pennsylvania contribute to the nutrient loading problems affecting of other states and industries in the Chesapeake Bay watershed. One way to reduce potential water quality problems from manure is to apply it to fields to help meet crop nutrient needs. However, the opportunity to jointly manage animal waste and crop nutrients as part of a single operation has decreased as a result of the trend towards fewer, larger, and more specialized animal production units.

Recoverable manure nitrogen increased by 20 percent over the 1982-1997 period.

In addition, in this period, more animals were being raised on farms with little land. As a result, manure nitrogen in “excess” of potential on farm use increased by 64 percent. For farms with livestock and limited amounts of cropland, field applications of manure may be done for disposal purpose of, rather than as part of a nutrient management plan. This situation can lead to applications of nitrogen and phosphorus at rates that are beyond what vegetation can utilize, leaving the excess to run off to surface water or to leach to ground water. Today, 60 percent of the manure nitrogen and 72 percent of the manure phosphorus generated by CAFO animals are still in excess of the farms' potential use. The total excess of phosphorus is a huge ½ billion tons.

It is these “excess” nutrients that probably pose the greatest risks to environmental quality. Thus, they require very careful management. The challenge is to convert that management effort into an opportunity with market value. The embodiments of this invention are intended to provide a range of value-added, damage-reducing options for nutrient management, which will benefit CAFO businesses, the adjacent communities, the surrounding environments, and adversely affected watersheds and water resources.

The geographic concentration of animal feeding operations can overwhelm the ability of a watershed to assimilate the nutrients contained in the waste and maintain water quality. If those excess nutrients from animal feeding operations could serve the fertilization needs of remote cropland and pastureland, then one approach to disposal is to relocate that animal waste for constructive disposal. Better yet, if the nutrients could be converted to a more easily transported form on the farm, the increased value might generate revenues that would reduce or offset the waste management costs. Alternatively, the conversion of the excess nitrogen and phosphorous into a slow-release compound where availability may be chemically managed within solubility limits would permit land-based disposal with little negative consequence. The simultaneous reduction or elimination of the public nuisance aspects would have further social value. Examples of regions with significant risk of water quality degradation from both nitrogen and phosphorous in manure include: Chesapeake Bay, Fruitful Rim, Prairie Gateway, Eastern Uplands, and Southern Seaboard. All states with significant CAFO operations have a stake in solving the problem of farm nutrient management.

Animal Market Growth

Despite the financial pressures facing many of our livestock and poultry producers, there still seems to be a great deal of optimism about the potential for growth in these sectors of the agriculture industry. This growth would follow a period in which there already has been a significant increase in the size and intensity of many livestock and poultry enterprises. Future development very likely would continue this trend toward large operations.

New Federal Regulations (December 2002)

The EPA was forced to issue the new anti-pollution rule by a federal court order as part of a lawsuit filed by an environmental group more than a decade ago. The new regulations mean so-called “factory farms” must apply for EPA and State water runoff permits by 2006, develop plans to handle manure and wastewater, and file annual reports with the government.

“It will help reduce what has been a growing problem—the fact that animal waste generated by CAFOs poses an increasing threat to the health of America's waters,” EPA Administrator Christine Todd Whitman said in a statement.

State governments will determine the types of permits to be issued to factory farms. The states will also have “substantial flexibility” and can authorize alternative performance standards for large livestock farms, the EPA said.

The Bush administration issued new rules on Dec. 16, 2002 requiring “factory farms” raising huge numbers of cows, pigs and chickens to curb manure runoff, but environmental groups said the plan did too little to protect streams and waterways (USA Today, Dec. 17, 2002). The details of the regulations are sketchy, but the rules were expected to force farmers to do more to prevent leaks and spills from man-made lagoons commonly used to catch animal wastes at mammoth livestock feeding operations. The Environmental Protection Agency and Agriculture Department scheduled a news conference on Monday afternoon to announce the new rules.

Farm groups have complained that it might cost $1 billion a year to comply with stricter water pollution requirements. The EPA was required to issue the regulation by a federal court order as part of a 1989 lawsuit filed by an activist seeking to protect the environment. Some 13,000 CAFOs would be required to obtain permits under the rules. A CAFO has at least 1,000 head of feeder cattle, 2,500 hogs or 30,000 broilers under one EPA definition. The rules would cover manure handling and require nutrient management plans and additional record-keeping by producers. EPA was likely to alter a proposal for “zero discharge” of pollutants from CAFOs to apply to new facilities, but not existing barns, feedlots and broiler houses.

Regulatory Controversy

The controversy in this issue is that many Environmental groups felt that the Bush administration did not go far enough to protect water and air. The new rules still allow rudimentary open-air lagoons and the land application of animal wastes, even though North Carolina and some other states have banned such systems on new farms, according to Environmental Defense. “Factory farms discharge a staggering amount of contaminants into the atmosphere, and the EPA regulations fail to seriously address air emissions and their well-documented impacts on public health and water quality,” (Dan Whittle, senior attorney with Environmental Defense Fund). Some environments feel that the new rules are a major step backward because they do not regulate air emissions (CO₂, H₂S, and NH₄ and other gasses).

During the past decade, factory farms (CAFOs) have come to dominate U.S. production of beef, pork and poultry. The National Academy of Sciences, recently, called for the EPA to adopt better ways to estimate air pollution and greenhouse gases from factory farms. Local residents often complain about odor, particulate matter and hydrogen sulfide gas emitted from animal manure on large farms. Melanie Shepherdson of the Natural Resources Defense Council, which sued EPA in 1989 to force action on water pollution by livestock feeders, said the new rules would be weaker than a version proposed by the Clinton administration.

In addition, the new regulations are not expected to require large meatpackers to be listed with farmers who are co-holders of CAFO permits. Without a co-permittee approach, meatpackers do not face liability for problems on the farm, activists said. Although producers will be required to write plans for manure management, the rules would allow growers to keep the documents on the farm and make it difficult to know if producers were following their plans.

Environmentalists also were concerned the rule would allow states to create a general permit for all CAFOs, rather than a public process for crafting a permit for each operation. They also were watching for EPA's decision on storage capacity for lagoons in cases of heavy rain. Spills can contaminate streams unless the catchments hold large amounts of rainwater.

With the impending CAFO rule in mind, Congress boosted finding for the USDA land stewardship programs by 80 percent in the new farm policy law enacted in May. Funding for the Environmental Quality Incentives Program, which shares with producers the cost for controlling manure run-off, was set at $900 million a year. It had been $200 million a year.

Waste or Resource?

While the question of whether manure is a waste or a resource does not rank with “to be or not to be” as one of life's burning issues, it is a very important consideration in manure management. People who deal with environmental issues like to use the phrase, “a waste is simply a resource in the wrong place.” When that resource gets into really the wrong place, it becomes a contaminant and creates pollution. Every year, we see examples of manure being a resource, a waste, and a pollutant. Since our society tends to dispose of wastes and manage resources, the way that people view and handle manure will be affected tremendously by the answer to this question.

Waste disposal is a cost to all businesses, including livestock and poultry operations. The focus of waste disposal, then, has been to minimize or avoid the cost to the business. This is the principle guiding much of our current manure disposal. The choice of methods used by businesses to achieve low-cost disposal has been guided mainly by societal pressures and environmental standards that are imposed on CAFOs.

On the other hand, envisioning and managing animal waste as a resource can result in a profit for waste byproducts that have value to society and the economy. Thus, the goal of waste management is to convert a liability economically into a resource with value and, in the process, reduce the cost of CAFO primary products. However, the agriculture system and industry have been slow to develop, demonstrate, bless, and publicize the necessary innovative approaches and technologies.

What are the limitations to using manure as a resource? Sadly, the product has real value to soil and plants, yet little or no cash value to farmers in its manure byproduct form. Manure is a bulky material that has relatively high moisture content and comparatively low concentrations of nitrogen, phosphorus, and potassium. It contains undesirable contaminants, has a high emission nuisance potential, and is not easy to apply at an optimum rate or location. However, these limitations can be overcome if the waste is converted to a more convenient form or if handling techniques are improved and if alternative fertilizer costs rise. Some sewage treatment plants have used magnesium hydroxide and calcium carbonate to limit wastestream release of phosphorous. Dairy farms have tried the combination to control lagoon odors. Others have tested the efficiency of magnesium hydroxide in capturing and settling phosphorous in lagoons. However, they focused on taming odor and overflow threats to air and watersheds.

So the answer to this question is that manure is not totally a waste or a resource right now. Our present handling practices range from operations where producers place great value on their manure and manage it scientifically and economically to other farms where the producers simply dump their manure on land as thickly as possible with little regard for societal and environmental impacts. Generally, though, manure is seen as a product that is too valuable to be considered a real waste, but not valuable enough to be a true resource. Its air emissions tend to rule out numerous uses regardless of the potential benefits and to prevent the conversion and distribution of the manure and its derivative products to sites where it is valued, constructively used, and not offensive or environmentally adverse. The present invention in its numerous embodiments addresses these limitations and the negative nutrient loading effects of manure on our watersheds, our watershed industries, and the public health and welfare.

### BRIEF SUMMARY OF THE INVENTION

The present invention is predicated on the successful treatment of sewage waste with magnesium hydroxide and/or calcium carbonate, dramatically reducing ammonia volatilization from the human and animal and vegetable waste being treated. Results also indicate that magnesium hydroxide and calcium compounds effectively reduce soluble phosphorus levels. The present invention is to apply controlled amounts of magnesium hydroxide and/or calcium carbonate with and without a carrier medium to animal litter in situ, to manure piles and animal waste and compost removed from animal enclosures, to animal waste in storage containers, and as an emissions controlling process in conjunction with manure and litter processing, manure-based and litter-based fertilizer production, manure and compost storage, and animal growing. A consequential effect is to reduce air emissions from fresh and stored animal waste and related fertilizer products by retaining and binding nitrogen and phosphorous from manure and litter in a concentrated, slow-release compound that improves the effectiveness of waste management and reduces farm runoff. Animal litter is composed of a mixture of bedding material, manure, spilled food and hair and/or feathers.

To retain more nitrogen in concentrated compound or fertilizer form, magnesium hydroxide and/or calcium carbonate with and without a carrier medium is applied to animal litter or manure in an amount sufficient to maintain the litter or manure pH at values which inhibit ammonia volatilization. Since animal litter and manure are highly variable in composition, the actual respective quantity and mix of magnesium hydroxide and calcium carbonate needed to inhibit volatilization may vary to as much as 25% of the litter weight (and higher for the manure weight) depending upon the water content of the waste. Alternatively, the amount required is based on the target molar ratio of Mg: NH₄: PO₄ being greater than 1:1:1, with the pH about 8.0. The magnesium hydroxide and calcium carbonate also retains and binds the nitrogen otherwise lost through volatilization plus that which normally would have remained, thereby increasing the available nitrogen retained and bound for possible fertilizer use and greatly reducing the nitrogen and phosphorous farm run-off threat. A carrier medium, if used, facilitates more rapid and more complete penetration of the litter and the manure by the magnesium hydroxide and/or calcium carbonate it carries, further reducing the immediate emissions potential and volatilization, and more quickly and completely extracting and binding the nutrients in concentrated, slow-release compound or fertilizer form. The resulting crystalline material releases nitrogen and phosphorous very much more slowly than plain manure or lagoon wastewaters with their high levels of dissolved, available nitrogen and phosphorous.

Naturally occurring calcium compounds also are added to reduce phosphorus solubility in the animal litter, animal waste, CAFO lagoons and pits, and manure in accordance with the present invention.

In one embodiment for the treatment of litter, magnesium hydroxide (and calcium carbonate or calcium compounds) in a carrier medium is applied to litter in the animal-growing enclosure prior to introduction of the animals.

In one embodiment for the treatment of litter, magnesium hydroxide (and calcium carbonate or calcium compounds) without a carrier medium is applied to litter in the animal-growing enclosure prior to introduction of the animals.

In another embodiment, the magnesium hydroxide (and calcium carbonate or calcium compounds) in a carrier medium is applied as periodically needed to the litter while the growing animals still reside in the enclosure.

In another embodiment, the magnesium hydroxide (and calcium carbonate or calcium compounds) without a carrier medium is applied periodically as needed to the litter while the growing animals still reside in the enclosure.

In another embodiment, the magnesium hydroxide (and calcium carbonate or calcium compounds) in a carrier medium is applied to litter and manure after removal of the grown animals.

In another embodiment, the magnesium hydroxide (and calcium carbonate or calcium compounds) without a carrier medium is applied to litter and manure after removal of the grown animals.

In another embodiment, the magnesium hydroxide (and calcium carbonate or calcium compounds) in a carrier medium are applied to litter or manure after it has been removed from the growing enclosure for temporary or long-term storage in any type of container, containment device or containment facility, including lagoons and pits.

In another embodiment, the magnesium hydroxide (and calcium carbonate or calcium compounds) without a carrier medium are applied to litter or manure after it has been removed from the growing enclosure for temporary or long-term storage in any type of container, containment device or containment facility, including lagoons and pits.

In another embodiment, litter or manure is treated with magnesium hydroxide (and calcium carbonate or calcium compounds) in a carrier medium prior to field application as a fertilizer or as a method of waste management or disposal.

In another embodiment, litter or manure is treated with magnesium hydroxide (and calcium carbonate or calcium compounds) without a carrier medium prior to field application as a fertilizer or as a method of waste management or disposal.

In yet another embodiment, magnesium hydroxide (and calcium carbonate or calcium compounds) in a carrier medium is applied to the litter or manure as part of the conversion of the litter or manure into fertilizer product for packaging and commercial sale in a form that has greatly reduced signature emissions of litter and manure.

In yet another embodiment, magnesium hydroxide (and calcium carbonate or calcium compounds) without a carrier medium is applied to the litter or manure as part of the conversion of the litter or manure into fertilizer product for packaging and commercial sale in a form that has greatly reduced signature emissions of litter and manure.

In another embodiment, the CAFO facilities of dairy and hog farms are washed down with a slurry of magnesium hydroxide (and calcium carbonate or calcium compounds) with a carrier medium that together with the waste is stored temporarily in a lagoon, pit or tank.

In another embodiment, the CAFO facilities of dairy and hog farms are washed down with a slurry of magnesium hydroxide (and calcium carbonate or calcium compounds) without a carrier medium that together with the waste is stored temporarily in a lagoon, pit or tank.

In another embodiment, the magnesium hydroxide and calcium compounds with a carrier medium are applied directly to the lagoons, pits, or tanks containing and receiving waste from CAFO enclosures.

In another embodiment, the magnesium hydroxide and calcium compounds without a carrier medium are applied directly to the lagoons, pits, or tanks containing and receiving waste from CAFO enclosures.

Additional features and advantages and variations of the present invention are further described, and will be apparent from the detailed description and from the preferred embodiments.

### BRIEF DESCRIPTION OF THE SEVERAL VIEWS OF THE DRAWING

FIG. 1. Struvite Crystals Are Formed from Chemistry Reaction of Magnesium Hydroxide and Calcium Carbonate with Ammonia and Phosphate.

Struvite crystals, which incorporate nitrogen and phosphorous, can reduce air emissions and nutrient run-off from animal waste via the current invention.

### DETAILED DESCRIPTION OF THE INVENTION

Application of Magnesium Hydroxide and/or Calcium Carbonate with or without a Carrier Medium to Animal Wastes, Litter, and Manure to Reduce Air Emissions and Retain Nitrogen and Control Phosphorous:

In the present invention and embodiments, Mg(OH)₂ and/or CaCO₃ with and without a carrier medium are applied for the prevention and control of air emissions from animal wastes, animal litter and animal manure from various species of animals that are farmed or raised for commercial market value including the major species in animal husbandry (avian, swine, bovine, reptilian, and pet animal species) as well as pet animals (e.g., cats, dogs, and zoo animals). The application may be direct or via a medium including pre-treated litter or with another material including a surfactant.

Mg(OH)₂ and/or CaCO₃ with and without a carrier medium is applied either as a dry powder or a liquid slurry to animal wastes, animal litter and or animal manures for the control of air emissions produced from an array of chemical and biological degradation and oxidation processes as the animal wastes decompose, dehydrate and or age over time. The appropriate mixing rate for dry powder depends upon the purity of the of Mg(OH)₂ and CaCO₃ mixture, the moisture content of the material being treated to control air emission, and the degree and efficiency of the mechanical mixing applied. Experimental results suggest that the application rates ranges from 50 lbs/ton to 300 lbs/ton.

Nitrogen and phosphorous as ammonia and phosphate in the presence of magnesium hydroxide and calcium compounds can combine to form a hard crystalline deposit called struvite when the molar ratio of Mg: NH₄: PO₄ is greater than 1:1:1, and the pH is 8.0-9.5. The struvite settles and can be recovered and used as a slow-release fertilizer or, under various conditions, can be land-filled or spread on fields with little concern for adverse environmental effects.

In Dairy and Swine Lagoons:

The protocol is to measure the Concentrations in the surface waters of Total Phosphate and Ortho Phosphate and add enough Mg(OH)₂ and CaCO₃ to create at least a molar ratio of magnesium to phosphorus ranging from a ratio of 1.05:1 to 1.3:1 depending upon pH. Studies at the University of Tennessee (Burns et al., 2001) have shown that a 90% reduction in soluble phosphorus could be attained via the precipitation of struvite crystals in a 140,000 Liter swine-slurry holding pond under field conditions.

In Poultry Litter:

The protocol is to use estimates of the average concentration of nitrogen and phosphorus in the chicken litter and then dose for these concentrations to convert them into struvite crystals. The following data have been developed as representative data for the residue levels in the litter at the end of a grow out period. Given that the number of birds in a given house can be upwards of 30,000 birds, an application dose rate has been developed for the estimated waste load expected per ton of expended litter at the end of the house clean out cycle (2 yrs). The nitrogen estimate used (40.8 g/kg of litter) is a residual in the litter after 2 years and underestimates the cycle available nitrogen because a large portion of the ammonia would have been released as an air emission into the house. Additional quantities of magnesium hydroxide and calcium compounds may be added for control of the air emissions that heretofore have escaped prior to the house clean-out. All calculations of requirements here have been based on phosphorus, balancing the limiting elements in forming struvite crystals, which would reduce ammonia evaporative loss (and chick exposure) and produce higher nitrogen levels in the litter at the end of 2 years with corresponding increase in the amount of struvite crystals formed.

The stoichiometric reaction requires that, for every part of ammonia to be removed, 1.736 mg/L of magnesium (Mg⁺²) and 2.21 mg/l of phosphorous (PO₄⁻³ —P) are required for each mg/L of ammonia converted to struvite crystals. Normally the phosphorus to nitrogen ratio in poultry litter ranges from ˜1:2 to 1:3. For a 2 year cleaning program, and a mean estimate of an average of 40.8 grams of total nitrogen produced/kg of litter, the suggested treatment would be from 3.4 to 5.1 tons of tailored magnesium hydroxide and calcium compounds added as a powder (ground to 325 mesh), depending on the phosphate levels, to the litter when the litter is first placed in the house. This amount will last for 2 years and covert 90% of the phosphate to struvite crystals. Alternatively, it can be added throughout the two-year cycle to facilitate availability at the top of the crust.

Since magnesium and phosphorus are both more limited than nitrogen in chicken manure, the option is to maximize the precipitation of (PO₄⁻³—P) into struvite crystals. In laboratory reactor bench tests these dose application rates have been found to reduce ammonia levels 90%.

Mg(OH)₂ and/or CaCO₃ mixture with and without a carrier medium can also be applied as a liquid slurry for any of the above applications when percent moisture is not a problem. The most desired application rate is 6 lbs of Mg(OH)₂ and/or CaCO₃ mixture per one hundred gallons of water (and carrier where included) depending upon the degree of emission producing elements and constituents in the animal waste, present and future percent moisture levels, range in air temperature and storage conditions and rate of dehydration. Once mixed, the slurry can be introduced to the animal litter in situ or prior to application of the litter. When the rearing conditions or animal housing conditions are established, the powder or the slurry quantities required to control manure emissions and retain nitrogen and phosphorous will be adjusted to characteristics of the manures present. Alternatively, the powder or slurry or pre-treated litter may be repetitively added to litter/manure-generating setting for optimal control of emissions from animal litters and animal wastes in the CAFO quarters/bams/houses/cages as well as lagoons, pits tanks, and other storage areas.

Application of Mg(OH)₂ and/or CaCO₃ with and without a Carrier Medium to Animal Wastes that are Collected and Treated by Advanced Waste Treatment Technologies to Meet Water Quality Standards and Control Contaminant Discharges and Emissions and Runoff:

In the present invention and embodiments, Mg(OH)₂ and/or CaCO₃ with and without a carrier medium are applied for the prevention and control of air emissions and to increase settling and dewatering of solids, increase the efficiency of waste and nutrient degradation, BOD, and COD concentrations in animal wastes treatment systems, and/or control emissions and discharges from animal waste treatment plants to local waters. The application may be direct or via a medium or in conjunction with another material at any location and stage in the waste treatment process.

Mg(OH)₂ and/or CaCO₃ with and without a carrier medium are applied as a dry powder or a liquid slurry to animal waste treatment plants (AWTPs), for retention of nitrogen and phosphorous and the control of emissions produced from an array of chemical and biological degradation and oxidation processes and to expedite waste and nutrient degradation and settling and dewatering of solids. The appropriate mixing rate for dry powder depends upon the purity of the Mg(OH)₂ and CaCO₃ mixture, the moisture content of the material being treated to retain nutrients and control emissions, and the degree and efficiency of the mechanical mixing applied. Experimental results suggest that the application rates ranges from 50 lbs/ton to 300 lbs/MGD of wastewater.

Nitrogen and phosphorous as ammonia and phosphate in the presence of magnesium hydroxide and calcium compounds can combine to form a hard crystalline deposit called struvite when the molar ratio of Mg:NH₄:PO₄ is greater than 1:1:1, and the pH is 8.0-9.5. The struvite settles and can be recovered and used as a slow-release fertilizer.

In Dairy and Swine Lagoons:

The protocol is to measure the concentrations in the surface waters of Total Phosphate and Ortho Phosphate and add enough Mg(OH)₂ and CaCO₃ to create at least a molar ratio of magnesium to phosphorus ranging from a ratio of 1.05:1 to 1.3:1 depending upon pH. Studies at the University of Tennessee (Bums et al., 2001) have shown that a 90% reduction in soluble phosphorus could be attained via the precipitation of Struvite Crystals in a 140,000 Liter swine-slurry holding pond under field conditions.

In Poultry Litter:

The protocol is to use estimates of the average concentration of nitrogen and phosphorus in the chicken litter and then dose for these concentrations to convert them into struvite crystals. The following data have been developed as representative data for the residue levels in the litter at the end of a grow out period. Given that the number of birds in a given house can be upwards of 30,000 birds, an application dose rate has been developed for the estimated waste load expected per ton of expended litter at the end of the house clean out cycle (2 yrs). The nitrogen estimate used (40.8 g/kg of litter) is a residual in the litter after 2 years and underestimates the cycle available nitrogen because a large portion of the ammonia would have been released as an air emission into the house. Additional quantities of magnesium hydroxide and calcium compounds may be added for control of the air emissions that heretofore have escaped prior to the house clean-out. All calculations of requirements here have been based on phosphorus, balancing the limiting elements in forming struvite crystals, which would reduce ammonia evaporative loss (and chick exposure) and produce higher nitrogen levels in the litter at the end of 2 years with a corresponding increase in the amount of struvite crystals formed.

The stoichiometric reaction requires that, for every part of ammonia to be removed, 1.736 mg/L of magnesium (Mg⁺²) and 2.21 mg/l of phosphorous (PO₄⁻³—P) are required for each mg/L of ammonia converted to struvite crystals. Normally the phosphorus to nitrogen ratio in poultry litter ranges from ˜1:2 to 1:3. For a 2 year cleaning program, and a mean estimate of an average of 40.8 grams of total nitrogen produced/kg of litter, the suggested treatment would be from 3.4 to 5.1 tons of tailored magnesium hydroxide and calcium compounds added as a powder (ground to 325 mesh), depending on the phosphate levels, to the litter when the litter is first placed in the house. This amount will last for 2 years and covert 90% of the phosphate to struvite crystals. Alternatively, it can be added throughout the two-year cycle to facilitate availability at the top of the crust.

Since magnesium and phosphorus are both more limited than nitrogen in chicken manure, the option is to maximize the precipitation of (PO₄⁻³—P) into struvite crystals. In laboratory reactor bench tests these dose application rates have been found to reduce ammonia levels 90%.

Mg(OH)₂ and/or CaCO₃ mixture with and without a carrier medium can also be applied as a liquid slurry for any of the above applications when percent moisture is not a problem. The most desired application rate is 6 lbs of Mg(OH)₂ and/or CaCO₃ mixture per one hundred gallons of water (and carrier where included) depending upon the extent of air emission producing elements and constituents in the animal waste.

Application of Mg(OH)₂ and/or CaCO₃ with and without a Carrier Medium to Animal Litter, Wastes, and Manures that Are to Be Converted into Plant Fertilizers:

In the present invention and embodiments, Mg(OH)₂ and/or CaCO₃ with and without a carrier medium is applied for the prevention and control of air emission in animal wastes, animal litter and animal manure and retention of nitrogen and phosphorous for conversion together with the litter and/or manure via manufacture into marketable plant fertilizer. The application may be direct or via a medium or with another material including a surfactant at any location and stage in the fertilizer production process beginning at the litter and manure generation and continuing through collection, storage, transportation, production and packaging for distribution and sale or for use by a captive entity.

Mg(OH)₂ and/or CaCO₃ with and without a carrier medium is applied as a dry powder or a liquid slurry to animal wastes, animal litter and or animal manures for the control of air emission(s) and smells produced from an array of chemical and biological degradation and oxidation processes as the animal wastes decompose and dehydrate and are manipulated and treated to produce plant fertilizers for commercial markets. The appropriate mixing rate for dry powder depends upon the purity of the Mg(OH)₂ and/or CaCO₃ mixture, the loading of animal wastes, the moisture content of the absorbent material being treated to control air emission, and the degree and efficiency of the mechanical mixing applied. Experimental results suggest that the application rates for developing commercial plant fertilizers ranges from 50 lbs/ton to 300 lbs/ton.

Nitrogen and phosphorous as ammonia and phosphate in the presence of magnesium hydroxide and calcium compounds can combine to form a hard crystalline deposit called struvite when the molar ratio of Mg:NH₄:PO₄ is greater than 1:1:1, and the pH is 8.0-9.5. The struvite settles and can be recovered and used as a slow-release fertilizer or can be landfilled for disposal.

In Dairy and Swine Lagoons:

The protocol is to measure the concentrations in the surface waters of Total Phosphate and Ortho Phosphate and add enough Mg(OH)₂ and CaCO₃ to create at least a molar ratio of magnesium to phosphorus ranging from a ratio of 1.05:1 to 1.3:1 depending upon pH. Studies at the University of Tennessee (Burns et al., 2001) have shown that a 90% reduction in soluble phosphorus could be attained via the precipitation of struvite crystals in a 140,000 Liter swine-slurry holding pond under field conditions.

In Poultry Litter:

The protocol is to use estimates of the average concentration of nitrogen and phosphorus in the chicken litter and then dose for these concentrations to convert them into struvite crystals. The following data have been developed as representative data for the residue levels in the litter at the end of a grow out period. Given that the number of birds in a given house can be upwards of 30,000 birds, an application dose rate has been developed for the estimated waste load expected per ton of expended litter at the end of the house clean out cycle (2 yrs). The nitrogen estimate used (40.8 g/kg of litter) is a residual in the litter after 2 years and underestimates the cycle available nitrogen because a large portion of the ammonia would have been released as an air emission into the house. Additional quantities of magnesium hydroxide and calcium compounds may be added for control of the air emissions that heretofore have escaped prior to the house clean-out. All calculations of requirements here have been based on phosphorus, balancing the limiting elements in forming struvite crystals, which would reduce ammonia evaporative loss (and chick exposure) and produce higher nitrogen levels in the litter at the end of 2 years with corresponding increase in the amount of struvite crystals formed.

The stoichiometric reaction requires that, for every part of ammonia to be removed, 1.736 mg/L of magnesium (Mg⁻²) and 2.21 mg/l of phosphorous (PO₄⁻³—P) are required for each mg/L of ammonia converted to struvite crystals. Normally the phosphorus to nitrogen ratio in poultry litter ranges from ˜1:2 to 1:3. For a 2 year cleaning program, and a mean estimate of an average of 40.8 grams of total nitrogen produced/kg of litter, the suggested treatment would be from 3.4 to 5.1 tons of tailored magnesium hydroxide and calcium compounds added as a powder (ground to 325 mesh), depending on the phosphate levels, to the litter when the litter is first placed in the house. This amount will last for 2 years and covert 90% of the phosphate to struvite crystals. Alternatively, it can be added throughout the two-year cycle to facilitate availability at the top of the crust.

Since magnesium and phosphorus are both more limited than nitrogen in chicken manure, the option is to maximize the precipitation of (PO₄⁻³—P) into struvite crystals. In laboratory reactor bench tests these dose application rates have been found to reduce ammonia levels 90%.

Mg(OH)₂ and/or CaCO₃ mixture with and without a carrier medium can be applied as a liquid slurry for any of the above applications when percent moisture is not a problem. The most desired application rate is 6 lbs of Mg(OH)₂ and CaCO₃ mixture per one hundred gallons of water (and carrier where included) depending upon the extent of air emission producing elements and constituents in the animal wastes, animal litters, and animal manures present, the respective percent life cycle moisture levels, the range in air temperature and storage conditions, the rate of dehydration, and the characteristics of the process/treatment system being used to produce and store plant fertilizers.

Application of Mg(OH)₂ and/or CaCO₃ with a Carrier Medium for Better Penetration and More Effective Air Emissions Control

The effectiveness of liquid slurry application may be enhanced considerably by inclusion of a carrier which will transport the Mg(OH)₂ and CaCO₃ more completely and immediately through the manure.

The current invention includes optional use of a carrier selected on the following bases:

1. Its efficacy By utilizing a scientific method of “molecular stacking” it allows topical applications to work, by safely carrying necessary ingredients deep into the manure and litter rather than them sitting on the surface.

2. Low potential for toxicity due to its process, in which no metals combine to the molecular infrastructure. And also, the free carrier does not remain or penetrate through the membrane, but rather “back diffuses” meaning out of the membrane.

3. Unlimited Potential of uses Diversely soluble compounds can be stacked; therefore, oil (lipid) soluble and water soluble molecules can be placed in the same system without an emulsion or microemulsion.

4. The ease with which it can be produced and used.

A crown carrier is a preferred medium for the current invention; it is so named because many of its components when viewed as a three dimensional molecule from the side vantage point look like crowns. These systems are highly dependent on carbon, hydrogen, oxygen ratios for the most part. Some crown systems require the presence of other elements like nitrogen and phosphorous. The current invention prefers a carrier or ionotrope in which no metals are combined to the molecular infrastructure. This reduces the potential for metal toxicity.

There are several kinds of molecular systems that allow for the presentation, transfer and release of ‘inserted’ substances. Micelles, reverse micelles, laminel type combinations, hexarands, calixerands and crowns are just a few examples of these molecular systems. The vast majority of these systems are used for the transfer of metals or small molecules.

Jean-Marie Lehn in his book, Supramolecular Chemistry, explains that “Carrier-Mediated Transport (or facilitated diffusion) consists of the transfer of a substrate across a membrane, facilitated by a carrier molecule located in the membrane. It is a cyclic process comprising four steps: (1) formation of the carrier-substrate complex at one interface; (2) diffusion of the complex through the membrane phase; (3) release of the substrate at the other interface; (4) back diffusion of the free carrier.” Importantly, through this mechanism, the free carrier does not remain or penetrate through the membrane, but rather ‘back diffuses’ meaning out of the membrane.

According to Lehn, carrier design requires suitability for the membrane and the system, a suitable hydrophilic-lipophilic balance (HLB), the ability to reach interface, and enter into contact with the aqueous phase. The carrier cannot be too bulky so as to allow for rapid diffusion and must bear functional groups that account for acid-base interactions. The complexity of carrier design requires that each system, whether skin, petroleum, or plant/animal materials, that is addressed is understood. The carrier design is dependent on the addressed system.

These kinds of Carrier-Mediated Transports have enormous application potential that will vastly improve the targeted system. To understand how to design a Carrier-Mediated Transport, it is essential to understand the targeted system and what substrate will create the desired change in that system. Carrier-Mediated Transports are not ‘pure’ in nature when more than one substrate (chemical component) is required to create the desired change.

The invention embraces several kinds of molecules that can act as “Host” for the respective “Guests” that need to be delivered to a specific system. These “Host” molecules are placed in a chemical environment with the predescribed HLB so as to allow for the molecular stacking of ‘Guest’ molecules that can be diversely soluble which will allow for release into lipid-aqueous systems.

The host molecules are formed from a specialized series of amphoterics and ethoxylates using a metallo salt as a catalyst. GC Mass Spectrum analysis indicates that the catalytic mix process has the expected end result of converting ethoxylates into long chain and aromatic based oxygen linked compounds.

Substrate molecules are placed within the open cavity (both circular and horseshoe in shape) of these kinds of compounds. Depending on mix order and pH, diversely soluble compounds can be stacked due to electron attraction created by partially shared charges found in these kinds of molecules. Therefore, oil (lipid) soluble and water-soluble molecules can be placed in the same system without an emulsion or microemulsion.

In the current invention, the substrate molecules which will be introduced via the medium and will be absorbed throughout the litter, manure, compost, or fertilizer mixture will include Mg(OH)₂ and/or CaCO₃ (and perhaps supplemental materials).

Insertion of Minerals and Desired Elements with a Carrier to Reengineer the Nutrient Balance and pH of Litter and Manure Used as a Raw Material Input to Designer-Fertilizer Manufacture

In addition, a carrier medium may be used to introduce minerals and elements for the purpose of rebalancing/designing the resulting fertilizer product(s) for particular target applications. Nitrogen, phosphorous, and potassium are the primary constituents which may be re-balanced from the basic manure composition 4-3-4. However, additional other minerals and elements may be added for special plant-life environments and applications.

The Process:

The ability of the Mg(OH)₂ and/or CaCO₃ mixture with and without a carrier medium to neutralize acids, to react with sulfides that produce objectionable emissions, to condition solids in wastes and wastewaters, to manage pH, or assist in nutrient retention and removal depends upon the waste volume, temperature, composition and strength, Cation ratios, the physical geometry of the application medium, and the collection and treatment system.

Generation of H₂S in animal wastes is a result of anaerobic conditions and resultant bacterial populations (see Yarnell, 2000). Production of gases from urea and production of other nitrogen byproducts are functions of pH, the rate of dehydration of the wastes, and the sorbent materials present to dry the wastes. Once the dosage ratio for the Mg(OH)₂ and CaCO₃ mixture has been established, the critical issue is how much combined material to add in relationship to waste production rates and the production of H₂S and other air emissions by the animal wastes.

Inclusion of the carrier as an option in the current invention and its embodiments facilitates thorough penetration of the litter, manure, and fertilizer to control air emissions, retain nutrients, and reduce farm runoff more effectively and completely compared to other methods currently available.

The process chemistry is summarized as follows:

The pathway for production of hydrogen sulfide without the presence of Mg(OH)₂ and CaCO₃ envisioned in the present invention and its embodiments is:

SO₄²⁻+Organic Matter (in presence of SRBs)→S²⁻

S²⁻+H₂O+CO₂→H₂S

H₂S+2O₂ (with Thiobacillus in the slime layer)→H₂SO₄

where SRBs are sulfate reducing bacteria

The typical air emission properties and toxicity of hydrogen sulfide that the present invention is designed to overcome are:

| H₂S (ppm) | Effects on Humans |
|---|---|
| <.00021 | Olfactory detection threshold |
| .00047 | Olfactory recognition threshold |
| 0.5-30 | Strong odor (rotten eggs) |
| 50-300 | Eye & respiratory injury |
| 300-500 | Life threatening (toxic) |
| >700 | Lethal |

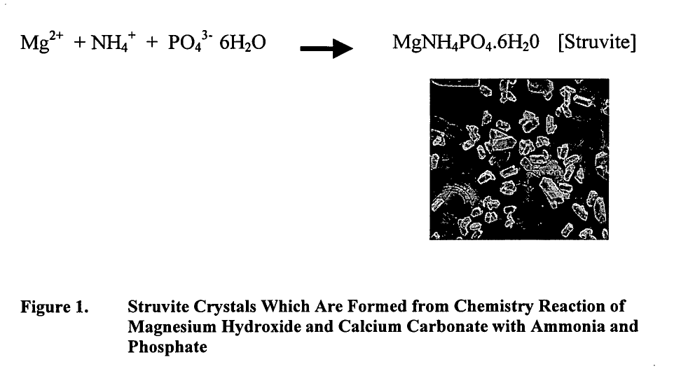

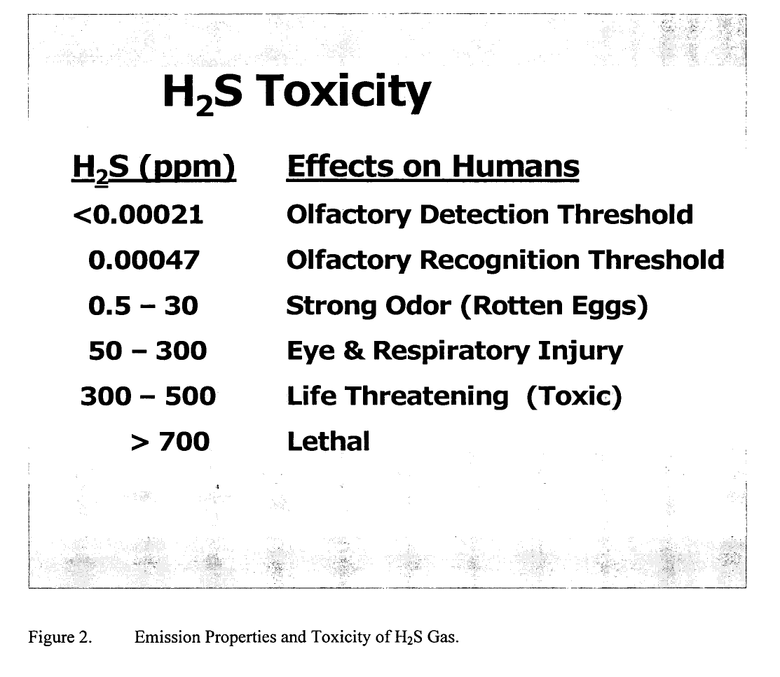

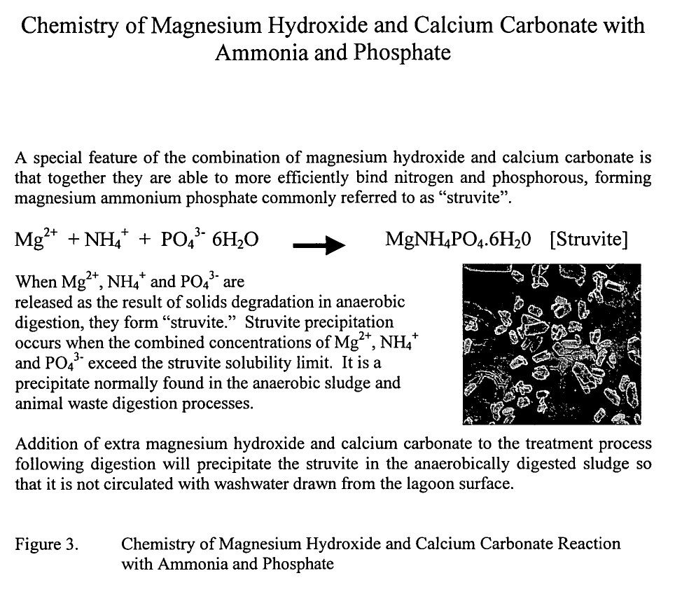

The chemical reaction of the animal waste with the magnesium hydroxide and calcium carbonate binds the nitrogen and phosphorous to produce a less soluble, slow release fertilizer component that reduces the undesired nutrient-loading of waterways and lakes and bays. Struvite (see FIG. 1) is a white crystalline substance consisting of magnesium, ammonium and phosphorus in equal molar concentrations (MgNH₄PO₄.6H₂O). Struvite forms according to the general reaction shown below:

Mg²⁺+NH₄⁺+PO₄³⁻+6H₂0 [symbol] MgNH4PO₄(H₂O)₆

The struvite crystal has a distinctive orthorhombic crystal structure and can be identified via X-ray diffraction (XRD) by matching the intensity and position of the peaks produced to a database for the crystal structure. Struvite precipitation can be separated into two stages: nucleation and growth. Nucleation occurs when constituent ions combine to form crystal embryos.

Crystal growth continues until equilibrium is reached and in systems continuously replenished with struvite constituents: e.g. wastewaters, and crystal growth continues indefinitely. Struvite precipitation is controlled by pH, supersaturation, temperature and the presence of impurities such as calcium and can occur when the concentrations of Mg²⁺, NH₄⁺ and phosphate ions pO³⁻ exceed the solubility product (Kₛₚ) for struvite. The Kₛₚ is given by the following expression:

Kₛₚ=[Mg²⁺][NH₄⁺][PO₄³⁻]

The relationship between Kₛₚ and pH indicates that struvite solubility decreases with increasing pH, which in turn leads to an increase in the struvite precipitation potential (SPP) of a wastewater. If the Ion Activity Product (IAP) (which is calculated by multiplying the equilibrium concentration of each ion) is greater than 7.08×10⁻¹⁴, a solution then is supersaturated with respect to struvite ions, and struvite precipitation is possible.

The formation and precipitation of struvite was first identified in wastewater treatment plants in 1939 as a scaling problem in pipes and screens, due to uncontrolled precipitation of struvite crystals in treatment works. Subsequent research has found that the formation and precipitation of struvite crystals can be managed with the driving force behind the formation of struvite being the presence of saturation levels of Mg²⁺, NH₄⁺ and PO³⁻ ions in solution with a pH ranging from 7.5 to 9.5. The recovered struvite can be sold as a fertilizer or disposed of by spreading it and turning it under in farm fields or other sites. The efficiency of forming struvite crystals as a means of phosphate removal in wastewaters has been reported in the literature to be over 90% indicating that struvite precipitation is an ideal way to reduce phosphate and nitrogen concentrations and recover nutrients from anaerobically treated wastes.

In swine waste pilot-scale treatment studies, ammonia concentrations have been reduced from 1500 mg/l to less than 10 mg/l in the final effluent. The optimum wastewater conditions are a pH of 8.0-9.5, and an ammonium: magnesium: phosphate molar ratio of 1:1.25:1. At higher pH's the struvite precipitation (Induction Time) can take a few hours; at lower pH's (7.0), it can take days. The induction time is affected by turbulence; doubling of the mixing speed or aeration (CO₂ removal with a subsequent rise in pH) cuts the Induction Time in half. The distinct advantage of dosing with a magnesium-based chemical is that the precipitation potential of the wastewater is increased and this will reduce the pH required to precipitate and recover struvite.

Struvite has been found to display excellent fertilizer qualities under specific conditions when compared with standard fertilizers. Qualities include its low solubility (slow release rates), and nitrogen and phosphorus components. Other factors that support the use of struvite as a fertilizer include the low heavy metal content of the product when compared to phosphate bearing rocks that are mined and supplied to the fertilizer industry. A factor that must be addressed is that struvite may require supplementation with potassium to meet the NPK (nitrogen: phosphorus: potassium) requirements of certain specific crops. Since supplies of phosphorus and the quality of phosphate bearing rock are decreasing, phosphorus recovery from animal wastes and wastewaters will become a significant source of phosphorus for agriculture fertilizers in the future.

FIG. 1 shows struvite crystals, which are formed from chemistry reaction of magnesium hydroxide and calcium carbonate with ammonia and phosphate. The struvite precipitate results from combining available nitrogen and phosphorous with magnesium hydroxide and calcium carbonate when they are introduced dry or wet into CAFO facilities to treat animal waste that is to be generated, as it is being generated, after it is generated, and when or after it is removed from generation locus. The reaction creates a compound which has potential fertilizer value and which when placed in fields or used as a fertilizer will release its nitrogen and phosphorous more slowly than untreated waste or waste treated with alum or PLT. Alternatively, the struvite compound can be land-filled for disposal. Use of a carrier medium facilitates the penetration of the waste by the magnesium hydroxide, calcium compounds, and any supplements being added to increase the value by adjusting the balance and pH of the resulting fertilizer product.

References:

Higgins, J. J., R. D. Meyers, N. M Sprague and K. Rarron. 1997. Controlling hydrogen sulfide in wastewater using base addition. Proceedings of the 70^{th} Annual Conference of the Water Environment Federation. Chicago, Ill.

Yarnell, E. M. 2000. Effect of Mg(OH)₂ Addition on Odor and Corrosion Associated with H₂S and the Effect on Waste Water Treatment Processes. Master Thesis. Bucknell University, Department of Civil and Environmental Engineering, May 11, 2000.

We claim:

1. A method for inhibiting ammonia volatilization in animal litter, comprising the steps of: admixing in situ magnesium hydroxide and/or calcium carbonate in a carrier medium to an animal litter composition sample comprising animal manure, bedding material, spilled food and hair or feathers in an amount sufficient to maintain a pH of the resulting litter composition sample at about 8.0.

2. A method as defined in claim 1, wherein said magnesium hydroxide and/or calcium carbonate in a carrier medium is admixed with the animal litter composition in an amount of from about 5% to about 25% by weight based on dry weight of said animal litter composition. Alternatively, the amount is based on the target molar ratio of Mg:NH₄:PO₄ being greater than 1:1:1, with the ph about 8.0.

3. A method for making nitrogen enhanced fertilizer based on animal litter comprising the steps of applying in situ a nitrogen-content-improving amount of magnesium hydroxide and/or calcium carbonate in a carrier medium to an animal litter composition comprising animal litter, bedding material, spilled food and hair or feathers, to be used for fertilizer, and thereafter further admixing magnesium hydroxide and/or calcium carbonate in a carrier medium and an animal litter composition.

4. A method as defined in claim 3, and further comprising the step of providing a sample of the magnesium hydroxide and/or calcium carbonate and animal litter composition wherein the amount of magnesium hydroxide in a carrier medium added is sufficient to maintain a pH value for the sample of the magnesium hydroxide and/or calcium carbonate and animal litter composition mixture after applying and mixing at about 8.0.

5. A method for inhibiting ammonia volatilization in animal litter, comprising the steps of:
(a) providing an enclosure for concentrated animal feeding;
(b) adding fresh bedding material to the enclosure;
(c) raising the numerous animals in the enclosure through growout;
(d) removing the mature animals after growout;
(e) applying in situ magnesium hydroxide and/or calcium carbonate in a carrier medium to a poultry litter sample comprising poultry manure, bedding material, spilled food and hairs/feathers in said enclosure in an amount sufficient to maintain a pH of the resulting magnesium hydroxide/litter sample at about 8.0; and
(f) admixing the carrier-mediated magnesium hydroxide and/or calcium carbonate in the sample.

6. A method as defined in claim 5, wherein steps (c) through (e) are repeated in sequence one or more times.

7. A method as defined in claim 5, wherein the magnesium hydroxide and/or calcium carbonate in a carrier medium is applied at the top of the litter and then mechanically admixed into the litter.

8. A method of inhibiting buildup of atmospheric ammonia in a commercial animal rearing facility which comprises applying in situ and admixing magnesium hydroxide and/or calcium carbonate in a carrier medium to animal litter in an amount sufficient to maintain the litter pH at values sufficiently low to inhibit the buildup of atmospheric ammonia in the animal rearing facility.

9. A method as defined in claim 8, wherein the magnesium hydroxide and/or calcium carbonate in a carrier medium is applied at the top of the litter and then mechanically admixed into the litter.

10. A method as defined in claims 8 and 9, wherein magnesium hydroxide and/or calcium carbonate in a carrier medium is applied and mechanically admixed into the litter at a site including a storage facility and manufacturing facility, remote from the animal rearing facility where the litter was generated.

11. A method for inhibiting ammonia and H₂S volatilization in animal litter compositions used in pet and zoo animal enclosures, comprising the steps of: admixing magnesium hydroxide and/or calcium carbonate in a carrier medium to natural bedding material (including wood chips or shavings or paper) for animals or to manmade bedding material (including fabrics and shapes manufactured from animal, vegetable, and spun-polymer fibers) for animals in an amount sufficient to maintain a pH value for the resulting litter composition sample of less than 8.0 until the neutralization capacity of the treated material is completely consumed by animal urination and defecation.

12. A method for thorough and efficient admixture of minerals and elements into litter and manure with a carrier to reengineer the nutrient balance and pH of the litter and manure used as a raw material input into designer-fertilizer manufacture.

13. A method for thorough and efficient admixture of minerals and elements into litter and manure without a carrier to reengineer the nutrient balance and pH of the litter and manure used as a raw material input into designer-fertilizer manufacture.

14. Methods as defined in claims 1 through 13, wherein calcium compounds (e.g., calcium carbonate) are included with the magnesium hydroxide.

15. A method for binding nitrogen and phosphorous in animal waste (Mg²⁺+NH₄⁺+PO₄³⁻6H₂O→MgNH₄PO₄.6H₂0), wherein magnesium hydroxide and calcium carbonate with a carrier medium are applied in dry form or slurry or spray (dry or wet) or in washwater to said animal waste in poultry houses, CAFO enclosures, and CAFO waste treatment facilities or lagoons, pits or tanks in advance of animal waste generation, during waste generation or after waste generation.

16. A method for binding nitrogen and phosphorous in animal waste (Mg²⁺+NH₄⁺+PO₄³⁻6H₂O→MgNH₄PO₄.6H₂0), wherein magnesium hydroxide and calcium carbonate without a carrier medium are applied in dry form or slurry or spray (dry or wet) or in washwater to said animal waste in poultry houses, CAFO enclosures, and CAFO waste treatment facilities or lagoons, pits or tanks in advance of animal waste generation, during waste generation or after waste generation.

17. A method as defined in claim 15 and 16, wherein the magnesium hydroxide and calcium compounds are applied to the animal enclosure floor or floor covering or litter or bedding before the animals are introduced or reintroduced.

18. A method as defined in claims 15 and 16, wherein the magnesium hydroxide and calcium compounds are applied while the animals present in the space or enclosure being treated with magnesium hydroxide and calcium compounds.

19. A method wherein magnesium hydroxide and calcium carbonate bind animal waste nitrogen and phosphorous (Mg²⁺+NH₄⁺+PO₄³6H₂O—MgNH₄PO₄.6H₂0) into a compound which settles, compacts, and is extractable with litter or manure or is recoverable from a lagoon bottom for processing into a fertilizer product.

20. A method as defined in claims 15 and 16 and 19, wherein MgNH₄PO₄.6H₂0 has settled to the waste lagoon bottom, permitting the lagoon surface water to be reused as a washwater or sprayed on fields for irrigation.

21. Methods as defined in claims 1 through 11 and 14 but without the use of a carrier medium.
